(19) United States
(12) Patent Application Publication
(10) Pub. No.: US 2006/0159724 A1
(43) Pub. Date: Jul. 20, 2006

(54) Nutritional supplement for the management of weight

(76) Inventor: Stacey J. Bell, Belmont, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/284,778

(22) Filed: Nov. 21, 2005

Related U.S. Application Data
(63) Continuation-in-part of application No. 11/166,904, filed on Jun. 23, 2005.
Application No. 11/166,904 is a continuation of application No. 10/361,838, filed on Feb. 6, 2003, now abandoned.
Application No. 10/361,838 is a continuation of application No. PCT/US01/24465, filed on Aug. 2, 2001.
Application No. PCT/US01/24465 is a continuation-in-part of application No. 09/634,246, filed on Aug. 8, 2000, now abandoned.
Application No. PCT/US01/24465 is a continuation-in-part of application No. 09/783,724, filed on Feb. 14, 2001, now abandoned.

(51) Int. Cl. A61K 47/00; A61K 31/522; A61K 31/405; A61K 31/353

(52) U.S. Cl. 424/439; 514/263.32; 514/456; 514/419

(57) ABSTRACT

Described herein is a low-glycemic nutritional supplement to be incorporated into the diet of an overweight or obese patient comprising low glycemic index ingredients including carbohydrate source, a source of protein, and a source of fat, and further comprising a source of green tea extract, a source of 5-hydroxytryptophan (5-HTP), and a source of chromium. The supplement provides active food-grade ingredients to improve the management weight loss, prevention of weight gain, and a feeling of satiety.

### RELATED APPLICATIONS

This application is a continuation-in part of U.S. application Ser. No. 11/166,904, filed Jun. 23, 2005, which is a continuation of U.S. application Ser. No. 10/361,838, filed Feb. 6, 2003 (now abandoned), which is a continuation of International Application No. PCT/US01/24465, which designated the United States and was filed on Aug. 2, 2001, published in English, which is a continuation-in-part of U.S. application Ser. No. 09/634,246, filed Aug. 8, 2000 (now abandoned) and U.S. application Ser. No. 09/783,724, filed Feb. 14, 2001, the entire teachings of which are incorporated herein by reference.

### BACKGROUND OF THE INVENTION

The prevalence of obesity in adults, children and adolescents has increased rapidly over the past 30 years in the United States and globally and continues to rise. Obesity is classically defined based on the percentage of body fat or, more recently, the body mass index (BMI), also called Quetlet index (National Task Force on the Prevention and Treatment of Obesity, Arch. Intern. Med., 160: 898-904 (2000); Khaodhiar, L. et al., Clin. Cornerstone, 2: 17-31 (1999)). The BMI is defined as the ratio of weight (kg) divided by height (in meters) squared.

Overweight and obesity are associated with increasing the risk of developing many chronic diseases of aging seen in the U.S. (Must, A. et al., JAMA, 282: 1523-9 (1999)). Such co-morbidities include type 2 diabetes mellitus, hypertension, coronary heart diseases and dyslipidemia, gallstones and cholecystectomy, osteoarthritis, cancer (of the breast, colon, endometrial, prostate, and gallbladder), and sleep apnea. It is estimated that there are around 325,00 deaths annually that are attributable to obesity. The key to reducing the severity of the diseases is to lose weight effectively. Although about 30 to 40% claim to be trying to lose weight or maintain lost weight, current therapies appear not to be working. Besides dietary manipulation, pharmacological management and in extreme cases, surgery, are sanctioned adjunctive therapies to treat overweight and obese patients (Expert Panel, National Institute of Health, Heart, Lung, and Blood Institute, 1-42 (June 1998); Bray, G. A., Contemporary Diagnosis and Management of Obesity, 246-273 (1998)). Drugs have side effects, and surgery, although effective, is a drastic measure and reserved for morbidly obese.

Since many of the drugs available to adults for combating obesity are not appropriate for children, only diet, exercise and behavior change are left as treatments. Some teens have reportedly sought out dangerous alternative products such as ephedrine, phenylpropanolamine (which was recently withdrawn), or potentially worse, smoking (Pray W. S., U.S. Pharmacist; 25:1-4 (2000); Tomeo C. A. et al., Pediatrics 104:918-924 (1999)).

Clearly new options are needed to help adults, children and adolescents lose weight and maintain the weight loss.

### SUMMARY OF THE INVENTION

The invention relates to a low-glycemic nutritional supplement that can help with the management of weight loss for overweight or obese patients which include adults, children and adolescent patients, the latter two being collectively referred to herein as “pediatric patients”. It should be understood that the nutritional supplement described herein and embodiments specified for pediatric use can be used for adults, children and adolescent patients. In embodiments specified for adults, it may not be medically recommended to give the adult formulation to a pediatric patient. Such administration is up to the medical professional.

The nutritional supplement comprises low-glycemic ingredients that together result in a low-glycemic product. The low-glycemic nutritional supplement comprises a low-glycemic ingredients comprising a carbohydrate source, a source of protein, fat and optionally source of dietary fiber. The low-glycemic-index nutritional supplement when administered to the patient, particularly pediatric patients, increases satiety, delays the return of hunger and decreases ad libitum food intake. The nutritional supplement comprises, for a 20 to 75 grams serving, from about 1 to about 75 grams low-glycemic-index carbohydrate (e.g., one or more low-glycemic-index carbohydrates that may further provide a source of dietary fiber), from about 1 to about 15 grams protein and from about 1 to about 20 grams fat. In one embodiment, the dietary fiber can be added to the nutritional supplement as part of the carbohydrate or as a separate ingredient.

The amount of dietary fiber is from about 0 to about 10 grams, with from about 4 to about 6 grams for a preferred snack serving and from about 6 to about 7 grams for a preferred meal serving. The ranges used herein are based upon a single serving. Two or more servings may be taken each day, especially for older children. This embodiment is particularly suited for pediatric patients but can be administered to adults.

In an embodiment suitable for adults, the nutritional supplement comprises a low glycemic index ingredients comprising a carbohydrate source, a source of protein, a source of fat, a source of caffeine and epigallocatechin gallate (EGCG) (e.g., provided in green tea extract), a source of 5-hydroxytryptophan (5-HTP), and a source of chromium. In preferred embodiments, the nutritional supplement comprises, for a 10-45 grams serving, from about 1 to about 25 grams carbohydrate (e.g., one or more low glycemic index carbrohydrates that may further provide a source of dietary fiber), from about 1 to about 10 grams protein, from about 1 to about 10 grams fat, from about 1 to about 1000 mg each of caffeine and epigallocatechin gallate from enough green tea to satisfy these needs, from about 1 to about 3000 mg 5-HTP, and from about 1 to about 2000 micrograms chromium (e.g., as chromium picolinate). The ranges used herein are based upon a single serving, where two servings are needed per day. Optionally, the nutritional supplement will contain caffeine if not adequately provided in useful quantities from the green tea extract.

The low-glycemic nutritional supplement, when administered to the pediatric patient increases satiety, delays the return of hunger and decreases ad libitum food intake. The nutritional supplement comprises, for a 20 to 75 grams serving, from about 1 to about 75 grams low-glycemic-index carbohydrate (e.g., one or more low-glycemic-index carbohydrates that may further provide a source of dietary fiber), from about 1 to about 15 grams protein and from about 1 to about 20 grams fat. The ranges used herein are based upon a single serving. Two or more servings may be taken each day, especially for older children.

The nutritional supplement can contain a carbohydrate source selected from the following: natural sweeteners (described below), fructose, barley, konjac mannan, psyllium and combinations thereof. The protein source is of a high biological value and is selected from whey protein concentrate, casein, soy, milk, egg and combinations thereof. The fat of the nutritional supplement is a non-atherogenic oil, preferably a vegetable oil comprising at least one vegetable oil selected from the group consisting of: canola, olive, soy, safflower, sunflower, corn and combinations thereof.

The nutritional supplement, additionally may comprise one or more of the following: micronutrients, dietary supplements, vitamins, minerals, flavoring, nutrients, and edible compounds, or a emulsifier.

The nutritional supplement can be made in a variety of forms, such as pharmaceutical compositions (e.g., tablet, powder, suspension, liquid, capsule, gel), nutritional beverages, puddings, confections (i.e., candy), ice cream, frozen confections and novelties, or baked and non-baked, extruded food products such as bars, chips and crackers. In another embodiment, the ingredients of the nutritional supplement can be administered separately, such as by incorporating certain components (e.g., bitter tasting ones) into a capsule or tablet and the remaining ingredients are provided as a powder or nutritional bar. The preferred form of the nutritional supplement is a nutritional beverage or a nutritional bar, such as a non-baked, extruded snack bar. The supplement can be formulated for single or multiple daily administrations, preferably twice daily, taken mid-morning and mid-afternoon, so as to control intake at the subsequent meal and satiety to appetite between meals or at night.

The invention further pertains to therapeutic methods for managing weight (controlling weight gain, preventing weight gain or promoting weight loss) for overweight or obese individuals. The nutritional supplement can be administered to an individual to aid in the feeling of satiety, increasing energy expenditure, fat burn and cause weight loss to be as fat rather than lean tissue. The nutritional supplement should be incorporated into a balanced deficit diet to effectively manage weight.

In a preferred embodiment, the nutritional supplement will comprise ingredients that are all natural and/or organic. All natural ingredients are recognized by those of skill to be free of artificial ingredients, colors, additives and preservatives. Use of all natural and/or organic ingredients will permit labeling of the nutritional supplement as such.

### DETAILED DESCRIPTION OF THE INVENTION

A description of preferred embodiments of the invention follows.

The invention pertains to a nutritional supplement for overweight and obese individuals, comprising carbohydrate source, a source of protein, optional dietary fiber which can be provided by the carbohydrate source or separately added, and fat; wherein the nutritional supplement has an overall low glycemic index. Based on clinical studies, the use of low-glycemic ingredients curb appetite and cause a reduction in daily caloric intake. As used herein the term “overweight” embraces obesity and is defined by commonly recognized clinical guidelines, such as BMI. These nutritional supplements when used with a weight loss program will facilitate weight loss and maintenance. The nutritional supplement may be in the form of an extruded bar or liquid, more preferably, a beverage or food.

The low-glycemic-index carbohydrate source can be provided by a single carbohydrate or a combination. The carbohydrate source can further provide a source of fiber and may be a natural sweetener, fructose, barley, konjac mannan, psyllium and combinations thereof. The protein source is of a high biological value and is selected from at least one of the following: whey protein concentrate, casein, soy, milk, egg and combinations of these. The fat is a non-atherogenic oil, preferably one of the following: canola, olive, soy, safflower, sunflower, corn and combinations of these. Additionally, the nutritional supplement may contain, micronutrients, vitamins, minerals, dietary supplements (e.g., herb), nutrients, emulsifiers, flavorings and edible compounds.

In a preferred embodiment, the nutritional supplement for overweight individuals comprises, for a 100 to 200 kcal/serving, with 130 kcal serving being most preferred, from about 1 to about 75 grams low-glycemic-index carbohydrate, from about 1 to about 20 grams protein and from about 1 to about 20 grams fat.

In yet another embodiment, the nutritional supplement comprises for a 120-125 kcal serving, from about 10 to about 30 grams low-glycemic-index carbohydrate, from about 1 to about 5 grams protein and from about 1 to about 5 grams fat.

In an embodiment, the nutritional supplement for adults comprises sources of carbohydrates, protein, fat, green tea extract, 5-HTP and chromium. Carbohydrates from low glycemic sources and fiber promote satiety by allowing glucose and insulin to be slowly released into the blood (e.g., barley, fructose, konjac mannan and psyllium). 5-HTP, which is a precursor of serotonin, increases satiety because it increases serotonin production, a hormone which stimulates satiety neurons in the hypothalamus. Green tea extract increases basal energy expenditure and fat oxidation, and chromium picolinate is added to promote the weight loss to be as fat rather than lean tissue. Caffeine increases the rate at which the body burns calories at rest. In the adult formulation, each serving of the product contains 45 kcal, and the breakdown of the macronutrient percentages is similar to the composition of the balanced calorie deficit diet (56% carbohydrate, 16% protein, and 27% fat) (Expert Panel, National Institute of Health, Heart, Lung, and Blood Institute, 1-42 (June 1998); Bray, G. A., Contemporary Diagnosis and Management of Obesity, 192-224 (1998)). The protein is a from high biological value source to promote protein synthesis (Crim, M. C. et al., Modern Nutrition in Health and Disease, ch. 2: 3-36 (1994)). Fat is from canola oil, which is non-atherogenic, and medium-chain triglycerides, which is oxidized immediately and unable to be stored (Babayan, V. K., Lipids, 22. 417-20 (1987)).

Use of the nutritional supplement is not intended to take the place of the prescribed diet, exercise, and medication regimen, recommended for overweight individuals; rather it works as an adjunctive therapy in those patients who are compliant with their healthcare providers' suggestions. The nutritional supplement can be made in a variety of forms such as a pharmaceutical composition (e.g., tablet, powder, suspension, liquid, capsule, gel), nutritional beverages, puddings, confections (i.e., candy), ice cream, frozen confections and novelties, or baked or non-baked, extruded food products such as bars, cookies and crackers, to assist patients with weight management.

The nutritional supplement can be formulated into a snack to be taken as part of the diet or it can be formulated as a meal replacement. For a snack, the nutritional supplement should provide from about 1 to about 250 kcal per serving; from about 20 to about 100 kcal being preferred; and from about 45 to about 50 kcal being most preferred. As a meal replacement, the nutritional supplement will provide from about 300 kcal to about 350 kcal per serving.

For the purposes of this invention, a preferred nutritional supplement comprises the components described above as a single serving (serving unit), whereby one or a plurality (preferably, two) of these supplement(s) is(are) consumed daily. The proportions of these ingredients are based on a 45 gram serving for adults or a 27 gram serving for pediatric patients. Two servings (e.g., 8 oz. water with 10-15 g powder) should make up 90 kcal and 1 bread exchange for adults, and 240-250 kcal for pediatric patients. In a preferred embodiment, each serving (serving size) contains 45 kcal for an adult, and 120-125 kcal for a pediatric patient and is comprised of macronutrient percentages in concert with the dietary recommendations of the American Diabetic Association and American Dietary Association. Other serving sizes are contemplated in the invention. The total amount of each ingredient should be appropriately adjusted.

The use levels for ingredients incorporated into the nutritional supplement are illustrated in Tables 1 (for a beverage) and 2, and represent broadest, preferred and most preferred embodiments.

TABLE 1
Adult Patients - Based upon 10-15 g serving size of powder in 8 oz. of water

| Nutrients | | | Recommended |
|---|---|---|---|
| (per serving) | Optimal Amount | Preferred Range | Range |
| Carbohydrate | 7 g total | 2-10 g total | 1-25 g total |
| | 1 g konjac | 0.5-5 g konjac | 0.5-11 g konjac |
| | 2 g fructose | 0.5-5 g fructose | 0.5-11 g |
| | 1 g barley | 0.5-5 g barley | fructose |
| | 1 psyllium | 0.5-5 g psyllium | 0.5-11 g barley |
| | 2 g other (e.g. | | 0.5-11 g |
| | flavorings, colors) | | psyllium |
| Protein | 2 g | 1-5 g | 1-10 g |
| Fat | 1.5 g | 1-5 g | 1-10 g |
| Green Tea Extract | Enough to contain 75 mg | 25-100 mg | 1-1,000 mg |
| | caffeine, 187.5 mg | caffeine and 100-500 mg | caffeine and 1-1,000 mg |
| | catechins, of which 135 mg | EGCG | EGCG |
| | are epigallocatechin | | |
| | gallate (EGCG) | | |
| 5-hydroxytryptophan | 450 mg | 200-800 mg | 1-3,000 mg |
| Chromium | 100 μg | 50-1,000 μg | 1-2,000 μg |

TABLE 2
Pediatric Patients

| Nutrients | | | | | Most Preferred | |
|---|---|---|---|---|---|---|
| (per serving) | Optimal Amount | | Preferred Range | | Range | |
| Carbohydrate | 1-75 | g | 10-30 | g total | 21 | g total |
| | 2 | g other (e.g. | | | | |
| | | flavorings, | | | | |
| | | colors) | | | | |
| Protein | 1-50 | g | 1-5 | g | 3 | g |
| Fat | 1-20 | g | 1-5 | g | 3 | g |

The ingredients that make up the nutritional supplement are described in detail below and with regard to their relative role each contributes to therapeutic advantages of the invention.

Carbohydrates

An important macronutrient of the nutritional supplement is carbohydrate because it has the greatest influence on satiety and subsequent weight loss. As used herein, satiety, refers to the sensation of fullness between one meal and the next and satiation refers to a sensation of fullness that develops during the progress of a meal and contributes to meal termination. Foods with low-glycemic-indexes evoke a smaller rise in blood glucose and insulin and a higher glucagon concentration, which promote satiety and prevent weight gain better than those carbohydrate-containing foods with higher ones because they take longer to digest and to be absorbed than carbohydrates with high- glycemic-indices (Expert Panel, National Institute of Health, Heart, Lung, and Blood Institute, 1-42 (June 1998)).

The “glycemic index” is a system of predicting subsequent rises in blood glucose after ingestion of carbohydrate-containing foods (Anderson, J. S. et al., Modern Nutrition in Health and Disease, ch. 70: 1259-86 (1994); Wolever, T. M. S. et al., Am. J. Clin. Nutr., 54: 846-54 (1991); Wolever, T. M. S. et al., Diab. Care, 12: 126-32 (1990)). The glycemic index characterizes the rate of carbohydrate absorption after a meal. It is defined as the area under the glycemic response curve during a 2-hour period after consumption of 50 g of carbohydrate from a test food divided by the area under the curve of a standard, which is either white bread or glucose. The glycemic index carbohydrates have the highest peak circulating glucose in a 2 hour period following ingestion of food. Conversely, low-glycemic-index carbohydrates cause a lower peak glucose and smaller area under the curve.

Many factors determine the glycemic index of foods. These include carbohydrate type, fiber, protein and fat content and the method of preparation (overcooked foods evoke a higher response). Generally high-glycemic-index carbohydrates are highly refined, and have a relatively high amount of glucose or starch compared to lactose, sucrose or fructose. Also, they are low in soluble fiber. The inclusion of fiber is important due to the way fiber facilitates weight loss by forming a gel with the food in the stomach. This gelling action reduces the rate of gastric emptying and hence digestion rates which promote satiety. Other factors which affect satiety are the amount of carbohydrate, the complexity of the carbohydrate, and the other foods that are eaten simultaneously with the carbohydrate (e.g., fiber, protein, fat) (Ludwig, D. S., J Nutr., 130: 280S-3S (2000); Wolever, T. M. S. et al., Am. J. Clin. Nutr., 54: 846-54 (1991); Wolever, T. M. S. et al., Diab. Care, 12: 126-32 (1990)). Bread and potatoes raise blood glucose more than beans. Other foods containing no or non-digestible carbohydrate ingested at the same time as carbohydrates (e.g., fat, fiber and protein) reduces postprandial blood glucose and insulin levels (Wolever, T. M. S., et al., Am. J. Clin. Nutr., 54: 846-54 (1991)).

The hormonal profile created from consumption of low-glycemic-index carbohydrates and fiber is a low glucose and insulin response and a high glucagon response (Expert Panel, National Institute of Health, Heart, Lung, and Blood Institute, 1-42 (June 1998)). The opposite effect is seen with high-glycemic-index carbohydrates. In particular, there is a rapid decline in blood glucose concentrations following a meal of high-glycemic-index carbohydrates as a result of the extreme counter regulatory hormones that are activated to normalized high levels of circulating glucose. These high-glycemic-index carbohydrates promote the uptake of glucose into the muscle, prevent gluconeogenesis from occurring in the liver, and inhibit lipolysis, thereby denying the body access to two major fuels, glucose and fat. After consumption of high glycemic index carbohydrates, the hormonal state created is similar to what occurs with the lack of food for several hours, the decrease in blood glucose and free fatty acids that induce hunger.

In addition, the ingestion of high-glycemic-index carbohydrates is undesirable because, calorie for calorie, these carbohydrates elicit higher insulin levels and c-peptide excretion than low-glycemic-index carbohydrates. This functional hyperinsulinemia may promote weight gain by preferentially directing nutrients away from oxidation in the muscle and toward storage as fat.

Insulin response may be more important than the glycemic response in weight loss, although the two are highly correlated (Holt, S. H. A. et al., Am. J. Clin. Nutr., 661: 1264-76 (1997)). Some foods elicit a greater insulin response than glycemic response. Similarly, eating carbohydrate-rich and protein-rich foods at the same meal increases the postprandial insulin response (Slabber, M. et al., Am. J. Clin. Nutr., 60: 48-53 (1994)).

Consumption of low-glycemic-index carbohydrates promotes weight loss through energy intake regulation (Ludwig, D. S., J. Nutr. 130:280S-283S (2000); Roberts, S. R., Nutr. Rev. 58:163-169 (2000)). Increased satiety, a delay in return to a state of hunger and a decrease in food intake at a subsequent meal occurs with ingestion of low-glycemic-index carbohydrates.

The most powerful influence of low-glycemic-index carbohydrates appears to be in the reduction of energy intake at subsequent meals (Roberts S. R., Nutr. Rev. 58:163-169 (2000)). Energy intake averaged 29% more after consumption of high glycemic index carbohydrates compared to low-glycemic-index carbohydrates. Studies in which the effect of different glycemic indexed have on satiety and satiation have produced conflicting results. Many of the studies had design flaws such as short duration and variability of test diets (differences in energy density of palatability) to establish a true result.

The use of low-glycemic-index carbohydrates for weight reduction has been evaluated twice in the pediatric population (Ludwig, D. S. et al., Pediatrics 102:e26 (1999); Spieth, L. E., et al., Arch Pedi. Adolesc. Med, 154:947-951 (2000)). In the Ludwig study, twelve adolescent pubertal boys (mean age 15.4±1.4 years) were evaluated on three separate occasions. The subjects consumed identical test meals at breakfast and lunch that had low, medium or high-glycemic-index carbohydrates. Ad libitum food intake was determined 5 hours after lunch. Voluntary energy intake after the high-glycemic-index meal was 53% greater that after the medium-glycemic-index meal and 81% greater that after the low-glycemic-index mean. In addition, compared to the low-glycemic-index mean, the high-glycemic-index meal resulted in higher serum insulin levels, lower plasma glucagon levels, lower postabsorptive plasma glucose and serum fatty acid levels, and evaluation of plasma epinephrine. The area under the glycemic response curve accounted for 53% of the variation in voluntary food intake. The hormonal and metabolic changes, resulting from rapid absorption of glucose following the high-glycemic-index meal, were thought to promote excessive food intake in obese subjects.

The second study, conducted by Spieth et al. was designed to compare the long term effects of a low-glycemic-index diet with a low-fat diet on weight loss. Subjects were given diet instructions, based on which group they were assigned to and exercise and behavioral change information. Besides differences in glycemic index, the diets differed in the percentage of energy contributed by fat. For the low-fat group, the goal was to consume 25-30% of energy as fat and in the low-glycemic-index group 30-35%. Those in the low-glycemic-index group were told to eat to satiety, rather than being told to restrict intake of certain foods. Those in the low-fat diet group were directed to restrict intake of certain foods. Both body weight and BMI decreased significantly more in the low glycemic group, even after adjusting for age, sex, ethnicity, baseline BMI or body weight. Conversely, no change in BMI occurred in the low fat diet group.

Patients who also eat a diet rich in low-glycemic-index carboydrates will have the best results (Expert Panel, National Institute of Health, Heart, Lung, and Blood Institute, 1-42 (June 1998)). This diet should include foods rich in vegetables, fruits, and legumes, moderate amounts of protein and healthful fats, and decreased intake of refined grain products, potato, and concentrated sugars.

Based upon this understanding, the nutritional supplement should comprise one or more sources of carbohydrates having a low glycemic index and which may optionally serve the dual purpose of providing a source of fiber (e.g., fructose, inulin, agave, natural sweeteners (described below) uncooked corn starch, barley flakes (e.g., hulless), konjac mannan, psyllium and chicory root). In a preferred embodiment, the carbohydrate has a low glycemic index (e.g., fructose, barley, konjac mannan) and provides a source of fiber comprising about 0 to about 10 g carbohydrate per serving for an adult. Two servings per day are needed at this use level.

For pediatric patients, the carbohydrate has a low-glycemic-index and provides a source of fiber comprising about 1 to about 75 g carbohydrate per serving. Two servings per day are needed at this use level. The preferred range is about 10 to about 30 g of low-glycemic-index carbohydrate per serving, more preferably, about 21 g per serving.

Fructose is a preferred carbohydrate for sweetening the nutritional supplement. It is sweeter than ordinary table sugar (sucrose), derived from beet or cane sugars, and has a low glycemic index (GI=32). Taken as part of a meal, fructose produces a smaller incremental rise in plasma glucose level does sucrose, glucose, potato starch, or wheat starch. From about 1 g to about 10 g fructose can be used; with about 2 g fructose per serving being preferred.

Another preferred carbohydrate for sweetening the nutritional supplement is a natural sweetener such as, but not limited to, evaporated cane juice, inulin, agave, honey, maple syrup, brown rice syrup, malt syrup, date sugar, fruit juice concentrate, and mixed fruit juice concentrate. Natural sweeteners are desirable when “organic” and/or “natural” food labeling is desirable. Natural sugar sources embrace unrefined sugar, evaporated cane juice (also referred to as direct consumption sugar, dried cane juice, unrefined sugar), mulled cane, demerara, musovado and rapadura. It should be understood that natural sweeteners may have glycemic indices that are higher end of the low-glycemic index chart. Balancing a combination of carbohydrates will keep the resultant product within the low-glycemic parameters.

Dietary fiber that may be suitable for use in the invention includes but is not limited to cellulose, seeds, hemicellulose (e.g., bran, whole grains), polyfructose (e.g., inulin and oligofructans), polysaccharide gums (e.g., Larch Arabinogalactan), oatmeal, barley, pectins, lignin, resistant starches. Examples of suitable fiber sources include but are not limited to wheat bran, cellulose, oat bran, corn bran, guar, pectin, psyllium.

In preferred embodiments, it is desirable to incorporate barley (e.g., barley crisp) into the nutritional supplement as a carbohydrate and fiber source. Of all the grains, certain forms of barley have some of the lowest glycemic indexes. Pearled barley (GI=36) and cracked barley (GI=72) have lower glycemic indexes than sweet corn (GI=78), rolled barley (GI=94), and instant white rice (GI=128). Further, it is desirable to use barley with its bran still on it (referred to as “hulless barley”), so that the naturally occurring fiber remains. From about 1 g to about 10 g of barley per serving based upon a 10-15 g serving can be used; with about 1 g per serving being particularly preferred. Thus, it provides a low glycemic source of carbohydrate and a source of fiber (14%), both of which are advantageous in maintaining good glucose and weight control.

Konjac flour, which comes from a perennial tuber called Amorphophallus konjac, is a dietary fiber (90%) and a polysaccharide with a very high molecular weight. In addition, this glucomannan hydrocolloid has the ability to increase the viscosity of the intestinal fluid (digesta), thereby limiting the transport of glucose into the bloodstream (Vuksan, V. et al., submitted for publications, (2000)). Konjac mannan also has a low glycemic index, promoting weight loss by increasing satiety in obese and non-obese patients with type 2 diabetes.(Doi, K. et al., Progress in Obesity Research, ch. 80: 507-14, (1990)). In preferred embodiments, the nutritional supplement should provide from about 1 g to about 10 g konjac per serving; with about 1 g of konjac mannan being preferred.

Another good source of fiber for use in the invention is psyllium. Psyllium husk fiber is a viscous, mostly water-soluble fiber prepared from blonde psyllium seed (Plantago ovata). Psyllium, because it is a dietary fiber, promotes satiety and minimizes weight gain (Ludwig, D. S. et al., Modern Nutrition in Health and Disease, ch. 70: 1259-86 (1994)). It also has been shown to reduce blood lipid concentrations and blood glucose levels (Anderson, J. W. et al., Am. J. Clin. Nutr., 70: 466-73 (1999); Anderson, J. W. et al., Am. J. Clin. Nutr., 71: 1433-8 (2000); Anderson, J. W. et al., Am. J. Clin. Nutr., 71: 472-9 (2000)). Psyllium can be added in amounts of from about 1 g to about 10 g per serving based upon a 10-15 g serving. Preferably, each serving of the nutritional supplement contains 1 g of psyllium, and two servings are taken daily. This, in conjunction with other soluble fiber consumed through a healthy diet, will contribute to controlling appetite and weight gain.

Protein

Sources of protein can be any suitable protein utilized in nutritional formulations and can include whey protein, whey protein concentrate, whey powder, egg, soy protein, soy protein isolate, caseinate (e.g., sodium caseinate, sodium calcium caseinate, calcium caseinate, and potassium caseinate), animal and vegetable protein and mixtures thereof. When choosing a protein source, the biological value of the protein should be considered first, with the highest biological values being found in caseinate, whey, lactalbumin, soy, delactosed milk solids, egg albumin and whole egg proteins. These proteins have high biological value; that is, they have a high proportion of the essential amino acids.

In a preferred embodiment, the preferred protein is whey protein concentrate or other protein with a high biological value to promote protein synthesis (e.g., casein, soy, milk, egg) and provides about 1 to about 5 g protein per serving, with approximately 2 g protein per serving being preferred for adults. For pediatric patients, the nutritional supplement provides about 1 to about 50 g protein per serving. The preferred amount of protein is between 1 to 5 grams per serving, more preferable, approximately 3 g per serving for pediatric patients.

Fats and Oils

Sources of fats can include but are not limited to vegetable oil, (e.g., canola oil, corn oil, soybean oil, sesame seed oil, safflower oil, sunflower oil, evening primrose oil, peanut oil, cottonseed oil, high oleic sunflower oil, rapeseed oil, olive oil), fish oil (e.g., menhaden oil, sardine oil) and mixtures thereof, all of which are examples of long-chain triglycerides; and coconut oil, macadamia oil, palm oil, palm kernel oil, or mixtures thereof, all of which are examples of medium-chain triglycerides. Partially hydrogenated oils may also be used. Additional sources of long-chain triglycerides and medium-chain triglycerides are described in U.S. Pat. No. 4,703,062, the entire teachings of which are incorporated herein by reference. Medium-chain triglycerides can spare the glucose stored in the muscle as glycogen and have fewer calories per gram than long-chain triglycerides. The oils can be used in their natural states; alternatively, structured triglycerides, which can be either randomly re-esterified or specifically re-esterified, can be generated from two or more oils and used as a fat source. Structured triglycerides can contain long-chain triglycerides; medium-chain triglycerides; or both long-chain and medium-chain triglycerides.

The nutritional supplement can also contain a non-atherogenic oil in combination with medium-chain triglycerides or by itself, including but not limited to canola, olive, soy, safflower, sunflower and corn.

In a preferred embodiment, the nutritional supplement includes a fat source containing long-chain triglycerides (e.g., canola oil); in another preferred embodiment, the fat sources are provided in an amount sufficient to delay gastric emptying. The nutritional supplement for adults includes from about 1 to about 10 g fat, preferably as canola oil. For pediatric patients, the nutritional supplement includes from about 1 to about 20 g fat, preferably 1 g to 5 grams of fat, more preferably 3 grams of canola oil.

Green Tea Extract

Recently, green tea has been shown to induce weight loss (Dulloo, A. G. et al., Am. J. Clin. Nutr., 70: 1040-5 (1999); Juhel, C. et al., J. Nutr. Biochem., 11: 45-51 (2000)). The mechanism for increasing energy expenditure by green tea has been postulated to be its flavonoid, and more specifically its polyphenolic content (Dulloo A. G. et al., Am. J. Clin. Nutr., 70: 1040-5 (1999)). One class of these compounds, the catechins, have been shown to inhibit catechol O-methyltransferase (COMT), an enzyme that degrades norepinephrine. This inhibition allows norepinephrine to exert a prolonged influence on thermogenesis and fat metabolism. Both of these metabolic processes are controlled by the sympathetic nervous system via norepinephrine. The delay in degrading norepinephrine allows for it to remain in the sympathetic synaptic cleft longer and exert its effect. Caffeine also has an effect on norepinephrine by inhibiting phosphodiesterases and prolonging the life of cAMP in the cell. These actions coupled with the sustained effect of norepinephrine caused by epigallocatechin gallate (EGCG) affect thermogenesis (Dulloo, A. G. et al., Am. J. Clin. Nutr., 49: 44-5 (1989); Dulloo, A. G. et al., Am. J. Clin. Nutr., 70: 1040-5 (1999)). Although there are numerous catechins in green tea, EGCG is probably the most influential. This can not be obtained in appreciable amounts from any other food source. In addition, green tea extract is that it has been shown in vitro to inhibit gastric and pancreatic lipases by 37% (Juhel, C. et al., J. Nutr. Biochem., 11: 45-51 (2000)). One weight loss product on the market today (Orlistat) also induces weight loss by the same mechanism and it has been proven to be efficacious (Hill, J. O. et al., Am. J. Clin. Nutr., 69: 1108-16 (1999)).

The amount of green tea extract incorporated into the nutritional supplement should be that which provides from about 25 to about 100 mg caffeine and from about 100 to about 500 mg epigallocatechin gallate. The preferred embodiment of the invention contains enough green tea extract to provide about 75 mg caffeine and about 187.5 mg catechins, of which 135 mg are epigallocatechin gallate, of which two servings are needed per day. Caffeine can be incorporated into the nutritional supplement alone or in combination with green tea extract, particularly if the green tea extract does not provide adequate use levels of caffeine.

5 Hydoxytryptophan (5-HTP)

The brain neurotransmitter, serotonin, has an inhibitory effect on eating behavior (Cangiano, C. et al., Am. J. Clin. Nutr., 56: 863-7 (1992)). This neurotransmitter appears to influence both energy balance and the circadian patterns of eating (i.e., three times during the day) by activating satiety neurons in the medial hypothalamus. Serotonin seems to interact antagonistically with norepinephrine, resulting in decreased appetite and carbohydrate consumption. The availability of serotonin is contingent upon the conversion of tryptophan to 5-HTP.

Twenty-eight obese subjects (BMIs between 30 and 40 kg/m2) took 900 mg of 5-HTP, three times a day 30 minutes before meals. No other dietary restrictions were imposed during the first 6-week period and then an energy-restricted diet of 1,200 kcal was imposed during a second 6-week period. Significant weight loss was observed during both periods with the 5-HTP, but not in the control arm (5%; p<0.02). Carbohydrate intake decreased about 50% in the first period, and early satiety was observed in 100% of the subjects during the first period and 90% of them during the second phase. This work supports a role for amino acids influencing the regulation of food intake (Cangiano, C. et al., Am. J. Clin. Nutr., 56: 863-7 (1992)).

5-HTP can be derived from Griffonia simplicifolia and made into a 95% pure standardized extract. Although 5-HTP is preferred, tryphtophan can be used.

Each serving of the nutritional supplement contains from about 200 to about 800 mg 5-HTP with 450 mg per serving being preferred.

Chromium

Dietary chromium is an essential trace element involved in potentiating the action of insulin. Improvements in insulin utilization may lead to reductions in body fat. Correcting insulin resistance may also have a positive effect on muscle mass, by slowing its catabolism (Kaats, G. R. et al., Cur. Theor. Res., 57: 757-56, 1996). Even during significant weight loss, lean body mass can be preserved with chromium. Chromium picolinate does not promote weight loss, but rather, during weight loss, it seems to shift the composition of the weight loss in favor of fat rather than lean mass.

Doses of about 200 mg or 400 mg appear to be effective and chromium, as picolinate rather than bound to yeast, appears to be more effective. Each serving of the nutritional supplement of the invention should provide from about 50 to 1000 μg chromium per serving. The preferred amount of chromium for the nutritional supplement is approximately 100 μg of chromium picolinate per serving, with two daily servings being preferred. Alternatively, chromium chloride may be used, but the picolinate form is preferred due to its beneficial effects on fasting plasma glucose (FPG) concentrations. Since the nutritional supplement is intended to supplement the diet of an individual, the daily allowance of chromium provided by the invention is intended to be less than the efficacious amount. The difference in the amount of chromium will likely be made up from the diet (e.g., nutritional supplements, foods, pharmaceutical compositions), which may comprise an additional 200 μg per day.

Further Ingredients

The nutritional supplement can also contain other ingredients such as one or a combination of other vitamins, minerals, antioxidants, fiber (e.g., ginkgo biloba, ginseng) and other nutritional supplements. Selection of one or several of these ingredients is a matter of formulation design, consumer and end-user preference. The amount of these ingredients added to the nutritional supplements of this invention are readily known to the skilled artisan and guidance to such amounts can be provided by the RDA and DRI (Dietary Reference Intake) doses for children and adults. Vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacin amide; zinc sulfate or oxide; calcium pantothenate; copper gluconate; riboflavin; beta-carotene; pyridoxine hydrochloride; thiamin mononitrate; folic acid; biotin; chromium chloride or picolinate; potassium iodide; selenium; sodium selenate; sodium molybdate; phylloquinone; Vitamin D3; cyanocobalamin; sodium selenite; copper sulfate; Vitamin A; Vitamin E; vitamin B6 and hydrochloride thereof; Vitamin C; inositol; Vitamin B12; potassium iodide.

The amount of other ingredients per unit serving is a matter of design and will depend upon the total number of unit servings of the nutritional supplement daily administered to the patient. The total amount of other ingredients will also depend, in part, upon the condition of the patient. Preferably the amount of other ingredients will be a fraction or multiplier of the RDA or DRI amounts. For example, the nutritional supplement will comprise 50% RDI (Reference Daily Intake) of vitamins and minerals per unit dosage and the patient will consume two units per day.

Flavors, coloring agents, spices, nuts and the like can be incorporated into the product. Flavorings can be in the form of flavored extracts, volatile oils, chocolate flavorings (e.g., non-caffeinated cocoa or chocolate, or chocolate substitutes, such as carob), peanut butter flavoring, cookie crumbs, crisp rice, vanilla or any commercially available flavoring. Flavorings can be protected with mixed tocopherols. Examples of useful flavorings include but are not limited to pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, cherry oil, walnut oil, cinnamon oil, clove oil, or peppermint oil; peanut butter, chocolate flavoring, vanilla cookie crumb, butterscotch or toffee. In a preferred embodiment, the nutritional supplement contains berry or other fruit flavors. The food compositions may further be coated, for example with a yogurt coating, if it is produced as a bar.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain artificial sweeteners, e.g., saccharides, cyclamates, aspartamine, aspartame, acesulfame K, and/or sorbitol. Such artificial sweeteners can be desirable if the nutritional supplement is intended for an overweight or obese individual, or an individual with type II diabetes who is prone to hyperglycemia.

Manufacture of the Nutritional Supplement

The nutritional supplements of the present invention may be formulated using any pharmaceutically acceptable forms of the vitamins, minerals and other nutrients discussed above, including their salts. They may be formulated into capsules, tablets, powders, suspensions, gels or liquids optionally comprising a physiologically acceptable carrier, such as but not limited to water, milk, juice, sodas, starch, vegetable oils, salt solutions, hydroxymethyl cellulose, carbohydrate. In a preferred embodiment, the nutritional supplements may be formulated as powders, for example, for mixing with consumable liquids, such as milk, juice, sodas, water or consumable gels or syrups for mixing into other nutritional liquids or foods. The powdered form has particular consumer appeal, is easy to administer and incorporate into one's daily regimen, thus increasing the chances of patient compliance. The nutritional supplements of this invention may be formulated with other foods or liquids to provide premeasured supplemental foods, such as single serving bars or beverages, for example.

To manufacture such a beverage, the ingredients are dried and made readily soluble in water or other consumable liquids as described above. The beverage is a preferred nutritional supplement form due to its ability to aid in the sensation of satiety if consumed at least one half hour prior to meals.

To manufacture such a food bar, the dry ingredients are added with the liquid ingredients in a mixer and mixed until the dough phase is reached; the dough is put into an extruder and extruded; the extruded dough is cut into appropriate lengths; and the product is cooled.

For manufacture of other foods or beverages, the ingredients comprising the nutritional supplement of this invention can be added to traditional formulations or they can be used to replace traditional ingredients. Those skilled in food formulating will be able to design appropriate foods/beverages with the objective of this invention in mind.

The nutritional supplement can be made in a variety of forms, such as puddings, confections, (i.e., candy), nutritional beverages, ice cream, frozen confections and novelties, or baked or non-baked, extruded food products such as bars. The preferred form is a powder for a beverage or a non-baked extruded nutritional bar.

In another embodiment, the ingredients can be separately assembled. For example, certain of the ingredients (e.g., the bitter tasting ones) can be assembled into a tablet or capsule using known techniques for their manufacture. The remaining ingredients can be assembled into a powder or nutritional bar, as described herein. The two assembled forms comprise the nutritional supplement and can be packaged together or separately, such as in the form of a kit, as described below. Further, they can be administered together or separately, as desired.

Use of the Nutritional Supplement

Obesity is a heterogeneous group of conditions with multiple causes (Kopelman P. G., Nature, 404: 635-43 (2000)). Body weight is determined by an interaction of genetics, the environment, and energy balance (i.e., the relationship between energy intake and energy expenditure). Energy expenditure has several components. The major one, basal metabolism, accounts for up to two-thirds of the daily total energy needs (Bray, G. A., Contemporary Diagnosis and Management of Obesity, 35-67 (1998)). This includes energy to maintain body temperature, contracting smooth muscles of the heart and gastrointestinal tract, and mobilization of substances like food and oxygen across cell membranes. Another one-tenth of the energy expenditure is dissipated through the thermic effect of food (energy cost of digestion, absorption, and metabolism of food), which is reduced in obesity. Lastly, exercise (physical activity) contributes to energy expenditure, which represents about 20 to 50% of the total (Kopelman, P. G., Nature, 404: 635-43 (2000)).

The active ingredients in the nutritional supplement work to increase the body's rate of energy expenditure. Both green tea and caffeine increase the rate at which the body burns calories at rest. While losing weight from increased burning of calories and increased satiety, the addition of chromium causes the weight lost to be fat rather than muscle. 5-HTP, which is a precursor of serotonin, stimulates brain serotonin and causes decreased carbohydrate intake and weight loss. The macronutrient carbohydrate is specifically chosen based on their ability to manage blood glucose levels and increase satiety. Protein and fat create a product with balanced nutrients comparable to a balanced deficit diet.

The composition and dietary supplements of the invention are intended to be orally administered daily. Based on the serving size of 10-15 g powder in 8 oz. water or 35 g of an extruded bar for pediatric patients, the recommended dosage is twice daily. For example, if the supplement is in the form of a food bar or beverage, then the patient would consume one mid-morning and mid-afternoon, where hunger would cause overeating at the next meal. (See “Example 3” for a sample meal plan using the dietary supplement.) Older adolescents can eat more than one bar at a sitting. The recommended daily amounts of each ingredient, as described above, serve as a guideline for formulating the dietary supplements of this invention. The actual amount of each ingredient per unit dosage will depend upon the number of units daily administered to the individual in need thereof. This is a matter of product design and is well within the skill of the dietary supplement formulator.

The ingredients can be administered in a single formulation or they can be separately administered. For example, it may be desirable to administer the bitter tasting ingredients in a form that masks their taste (e.g., capsule or pill form) rather than incorporating them into the nutritional composition itself (e.g., powder or bar). Thus, the invention also provides a pharmaceutical pack or kit comprising one or more containers filled with one or more of the ingredients of the nutritional compositions of the invention (e.g., nutritional supplement in the form of a powder and capsules containing green tea and caffeine). Optionally associated with such container(s) can be a notice in the form prescribed by a government agency regulating the manufacture, use or sale of pharmaceutical or dietary supplement products, which notice reflects approval by the agency of manufacture, use of sale for human administration. The pack or kit can be labeled with information regarding mode of administration, sequence of administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a formulation or tablet. Agents assembled in a blister pack or other dispensing means is preferred.

Methods are described for providing low glycemic nutritional supplement to overweight and obese adults, children and adolescents, wherein the amounts of carbohydrate, protein and fat are sufficient to aid in the management of weight loss, preferably the supplement is in the form of a extruded bar (e.g., food) or in the form of a liquid (e.g., beverage).

Also described are methods for providing an individual with a nutritional supplement more preferably in the form of a liquid (e.g., beverage) that aids in the feeling of satiety, management of weight gain and promotes weight loss.

All references provided herein are incorporated by reference in their entirety.

### EXAMPLES

### Example 1

### Nutritional Supplement for Management of Weight

In one embodiment, the nutritional supplement is a beverage that provides 45 kcal/unit serving, where one unit serving is a 11 gram powder suspended in 8 oz. water, and is to be administered twice daily. The nutritional supplement has the following characteristics:

approximately 7 g carbohydrate: fructose (2 g), konjac flour (1 g) (Opta Food Ingredients, Bedford, Mass.), psyllium (1 g), barley (1 g); aspartame to sweeten; 2 g other from whey, 5-HTP, green tea;

approximately 2 g protein: preferably, whey protein concentrates. Soy, casein, or other high biological value proteins may be substituted to improve flavor; approximately 1.5 g fat: canola oil (about 1 g) and other (about 0.5 g) (e.g., whey);

approximately 386 mg green tea extract with 53 mg caffeine: should comprise 23 mg caffeine and 187.5 mg of catechins, of which 135 mg is epigallocatechin gallate (InterHealth, Benecia, Calif.);

approximately 450 mg 5-HTP: (InterHealth, Benecia, Calif.); and

approximately 100 μg chromium: as picolinate (AMBI/Nutrition 21).

### Eample 2

### Nutritional Composition of the Food Product

| Nutrient | Amount | kcal | % kcal | Notes |
|---|---|---|---|---|
| Carbohydrate | 7 g (2 g fructose, 1 g | 28 | 62 | Low-glycemic |
| | psyllium, 1 g konjac | | | carbohydrate sources |
| | mannan, 1 g barley) | | | and fiber to promote |
| | | | | satiety |
| Protein | 2 g (2 g whey | 8 | 18 | High biological value |
| | protein) | | | sources |
| Fat | 1.5 g (1 g canola oil | 13 | 30 | Monounsaturated fatty |
| | and 0.5 g from other | | | acids (non- |
| | sources) | | | atherogenic) and could |
| | | | | have medium-chain |
| | | | | trigylcerides (not |
| | | | | stored as fat) |
| Green tea extract | (75 mg caffeine, 135 mg | | | Shown to increase |
| | epigallocatechin | | | resting energy |
| | gallate, and 187.5 mg | | | expenditure |
| | catechins) | | | |
| 5-hydroxytryptophan | 450 mg | | | Shown to decrease |
| | | | | appetite |
| Chromium | 100 mcg | | | Shown to promote loss |
| picolinate | | | | of body fat instead of |
| | | | | lean tissue during |
| | | | | weight loss |

### Eample 3

### Weight Loss Meal Plan Using Food Product

A sample meal plan of a balanced deficit diet incorporating the food product is presented below. Women following this 1,200-kcal plan should take a multivitamin with minerals and calcium supplements to supply about 350 mg of elemental calcium.

Breakfast: Whole wheat bread, 1 slice; Jelly, 2 tsp., margarine, 1 pat; Cereal, shredded wheat, ½ cup Milk, 1%, 1 cup; Coffee, 1 cup

Mid-morning snack: Orange juice, ¾ cup containing the nutritional supplement

Lunch: Turkey sandwich—Whole wheat bread, 1 slice; Turkey, 2 oz.; Lettuce, 1 leaf; Tomato, 3 medium slices; Mayonnaise, low-calorie, 1 tsp.; Water, 1 cup

Mid-afternoon snack: Apple, 1 medium and the nutritional supplement

Dinner: Salmon, 2 ounces; Vegetable oil, 1½ tsp.; Baked potato, ¾ medium; Margarine, 1 tsp.; Green beans, seasoned with margarine, ½ cup; Carrots, ½ cup; Iced tea, unsweetened, 1 cup; Water, 2 cups

Evening Snack: Milk, 1%, ½ cup; Popcorn, 1 cup

### Example 4

### Nutritional Supplement for Management of Weight in Pediatric Patients

In one embodiment, the nutritional supplement is a beverage that provides 120-125 kcal/unit serving, where one unit serving is to be administered twice daily. The nutritional supplement has the following characteristics:

approximately 21 g carbohydrate;

approximately 3 g protein: preferably, whey protein concentrates; soy, casein, or other high biological value proteins may be substituted to improve flavor;

and approximately 3 g fat: preferably canola oil.

### Example 5

### Nutritional Composition of the Food Product for Pediatric Patients

| | | | | % | |
|---|---|---|---|---|---|
| Nutrient | Amount | | kcal | kcal | Notes |
| Carbohydrate | 21 | g | 84 | 69 | Low glycemic |
| | | | | | carbohydrate sources |
| | | | | | and fiber to promote |
| | | | | | satiety |
| Protein | 3 | g (3 g whey | 12 | 10 | High biological value |
| | | protein) | | | sources |
| Fat | 3 | g (canola oil) | 26 | 21 | Monounsaturated fatty |
| | | | | | acids (non- |
| | | | | | atherogenic) and could |
| | | | | | have medium-chain |
| | | | | | trigylcerides (not |
| | | | | | stored as fat) |

While this invention has been particularly shown and described with references to preferred embodiments thereof, it will be understood by those skilled in the art that various changes in form and details may be made therein without departing from the scope of the invention encompassed by the appended claims.

What is claimed is:

1. A low-glycemic nutritional supplement for overweight and obese individuals, comprising a carbohydrate source, a source of protein, optional dietary fiber and fat; wherein the amounts of carbohydrate, protein and fat are sufficient for use in individuals to aid in the management of weight loss.

2. The low-glycemic nutritional supplement of claim 1, further comprising a source of caffeine and epigallocatechin gallate, a source of 5-hydroxytryptophan (5-HTP), and a source of chromium.

3. The low-glycemic nutritional supplement of claim 1, wherein the nutritional supplement is in the form of a powder.

4. The low-glycemic nutritional supplement of claim 1, wherein the nutritional supplement is extruded.

5. The low-glycemic nutritional supplement of claims 1, wherein the carbohydrate source further provides a source of fiber.

6. The low-glycemic nutritional supplement of claim 1, wherein the carbohydrate source is selected from the group consisting of unrefined sugar, evaporated cane juice, honey, maple syrup, brown rice syrup, date sugar, fruit juice concentrate, mixed fruit juice concentrate, inulin, agave, fructose, barley, konjac mannan, psyllium and combinations thereof.

7. The low-glycemic nutritional supplement of claim 1, wherein the protein source is of a high biological value.

8. The low-glycemic nutritional supplement of claim 1, wherein the protein source comprises at least one protein source selected from the group consisting of whey protein concentrate, casein, soy, milk, egg and combinations thereof.

9. The low-glycemic nutritional supplement of claim 1, wherein fat is a non-atherogenic oil or a partially hydrogenated oil.

10. The low-glycemic nutritional supplement of claim 9, wherein the non-atherogenic oil is vegetable oil comprising at least one vegetable oil selected from the group consisting of: canola, olive, soy, safflower, sunflower, corn and combinations thereof.

11. The low-glycemic nutritional supplement of claim 10 wherein the non-atherogenic oil is canola oil.

12. The low glycemic nutritional supplement of claim 1, wherein the fat source is a mixture of canola oil and medium chain triglycerides (MCTs).

13. The low glycemic nutritional supplement of claim 2, wherein the source of chromium is in the picolinate form.

14. The low-glycemic nutritional supplement of claim 2, wherein the source of caffeine and epigallocatechin gallate (EGCG) is from green tea extract.

15. The low-glycemic nutritional supplement of claim 1, additionally comprising one or more of the following: micronutrients, dietary supplements, nutrients, edible compounds and flavorings.

16. A low-glycemic nutritional supplement for overweight and obese individuals comprising, for a 100 to 200 kcal/serving, from about 1 to about 75 grams low-glycemic-index carbohydrate, from about 1 to about 20 grams protein and from about 1 to about 20 grams fat; wherein the amounts of carbohydrate, protein and fat are sufficient for use in individuals to aid in the management of weight loss.

17. A low-glycemic nutritional supplement comprising, for a 120-125 kcal serving, from about 10 to about 30 grams low-glycemic-index carbohydrate, from about 1 to about 5 grams protein and from about 1 to about 5 grams fat, wherein the amounts of carbohydrate, protein and fat are sufficient for use in individuals to aid in the management of weight loss.

18. A low-glycemic nutritional supplement comprising, for a 45 kcal serving, from about 1 to about 25 grams carbohydrate, from about 1 to about 10 grams protein, from about 1 to about 10 grams fat, an amount of green tea extract providing about 1 to about 1,000 milligrams caffeine and about 1 to about 1,000 milligrams epigallocatechin gallate (EGCG), from about 1 to about 3,000 milligrams 5-hydroxytryptophan, and from about 1 to about 2,000 micrograms chromium.

19. A low-glycemic nutritional supplement comprising, for a 45 kcal serving, from about 2 to about 10 grams carbohydrate, from about 1 to about 5 grams protein, from about 1 to about 5 grams fat, an amount of green tea extract providing about 25 to about 100 milligrams caffeine and about 100 to about 500 milligrams epigallocatechin gallate (EGCG), from about 1 to about 3,000 milligrams 5-hydroxytryptophan, and from about 50 to about 1,000 micrograms chromium.

20. A food or beverage comprising the low-glycemic nutritional supplement of claim 1.

21. A pharmaceutical composition comprising the low-glycemic nutritional supplement of claim 1.

22. A method of providing an individual with low-glycemic nutritional supplementation that aids in the feeling of satiety, comprising administering to an individual in need thereof the low-glycemic nutritional supplement of claim 1.

23. A method of providing an individual with low-glycemic nutritional supplementation that aids in the management of weight gain and promotes weight loss, comprising administering to an individual in need thereof the low-glycemic nutritional supplement of claim 1.

24. A method of providing an individual with low-glycemic nutritional supplementation that aids in the prevention of weight gain, comprising administering to an individual in need thereof the low-glycemic nutritional supplement of claim 1.

25. A method for managing platelet adherence, comprising administering to an individual in need thereof the low-glycemic nutritional supplement of claim 1.
